(19) United States
(12) Patent Application Publication
(10) Pub. No.: US 2009/0030474 A1
(43) Pub. Date: Jan. 29, 2009

(54) Sensor Driven Gastric Stimulation for Patient Management

(75) Inventors: Charles R. Brynelsen, Menlo Park, CA (US); Mir A. Imran, Los Altos, CA (US); Jacob A. Bashyam, Santa Clara, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: INTRAPACE, INC., Mountain View, CA (US)

(21) Appl. No.: 12/145,430

(22) Filed: Jun. 24, 2008

Related U.S. Application Data
(60) Provisional application No. 60/947,267, filed on Jun. 29, 2007.

(51) Int. Cl. A61N 1/05

(52) U.S. Cl. 607/40

(57) ABSTRACT

Gastric stimulation systems and methods are provided for treating a patient, particularly by modifying behavior of the patient leading to excess weight loss. In some embodiments, such weight loss is achieved with a combination approach which includes two or more of the following: acute screening of the potential patients, gastric stimulation, induction of symptoms or specific behaviors and integration of patient management data into the treatment plan. Gastric stimulation is provided to portions of the gastrointestinal tract, particularly the stomach, with the use of at least one electrode. A variety of gastric stimulation systems may be used, including stimulators that are endoscopically placed, laparoscopically placed or placed by modified or combination methods.

### CROSS-REFERENCES TO RELATED APPLICATIONS

This application claims the benefit of under 35 U.S.C. §109(e) of U.S. Provisional Patent Application No. 60/947,267 filed on Jun. 29, 2007, the disclosure of which is incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Since the mid-seventies, the prevalence of obesity has increased sharply for both adults and children. Data from two National Health And Nutrition Examination Surveys (NHANES) show that among adults aged 20-74 years the prevalence of obesity increased from 15.0% (in the 1976-1980 survey) to 32.9% (in the 2003-2004 survey). The two surveys also show increases in overweight among children and teens. For children aged 2-5 years, the prevalence of overweight increased from 5.0% to 13.9%; for those aged 6-11 years, prevalence increased from 6.5% to 18.8%; and for those aged 12-19 years, prevalence increased from 5.0% to 17.4%.

These increasing rates raise concern because of their implications for Americans' health. Being overweight or obese increases the risk of many diseases and health conditions, including the following: hypertension, dyslipidemia (for example, high total cholesterol or high levels of triglycerides), type 2 diabetes, coronary heart disease, stroke, gallbladder disease, osteoarthritis, sleep apnea and respiratory problems, and some cancers (endometrial, breast, and colon).

Obesity and its associated health problems have a significant economic impact on the U.S. health care system. Medical costs associated with overweight and obesity may involve direct and indirect costs. Direct medical costs may include preventive, diagnostic, and treatment services related to obesity. Indirect costs relate to morbidity and mortality costs. Morbidity costs are defined as the value of income lost from decreased productivity, restricted activity, absenteeism, and bed days. Mortality costs are the value of future income lost by premature death.

Electrical stimulation has been investigated as a treatment of obesity. Typically, such stimulation systems attempt to induce a desired outcome of reduced food intake and weight loss. However, many patients continue eating regardless of the electrical stimulation. Likewise, the human body is adept at becoming desensitized to continuous stimulation thereby reducing stimulation effectiveness over time.

Therefore, it would be desirable to provide an electrical stimulation system that is tailored to the needs of an individual patient, reduces the likelihood of desensitization, modifies behavior, and successfully leads to weight reduction. At least some of these objectives will be met with the present invention.

### BRIEF SUMMARY OF THE INVENTION

A gastric stimulation system is provided for treating a patient, particularly by modifying behavior of the patient leading to excess weight loss. In some embodiments, such weight loss is achieved with a combination approach which includes two or more of the following: acute screening of the potential patients, gastric stimulation, induction of symptoms or specific behaviors and integration of patient management data into the treatment plan. Acute screening removes non-responders to gastric stimulation from the patient population. Such patients are more suitably treated with other methodologies. Gastric stimulation is provided to portions of the gastrointestinal tract, particularly the stomach, with the use of at least one electrode. A variety of gastric stimulation systems may be used, including stimulators that are endoscopically placed, laparoscopically placed or placed by modified or combination methods.

Symptoms or specific behaviors are induced by gastric stimulation in response to sensed parameters in the body. A primary example of such a sensed parameter is ingestion. If the stimulation system senses that ingestion has occurred, it is then determined whether ingestion is desirable. Desirability of ingestion is based on one or more factors which will also be discussed in detail in later sections. If the ingestion is determined to be undesirable, stimulation is provided at a level at or above a “stop eating threshold” SET for the patient that typically causes the patient to feel a displeasurable sensation or symptom, such as gastric discomfort such as to the extent of nausea, pain or vomiting. Such a displeasurable sensation is one which causes the patient to stop the undesired ingestion, thus a specific behavior has been induced. Since each patient may react differently to the same level of stimulation, the SET will be customized for each patient by prior testing of the patient's response to gastric stimulation. If the patient does not stop the undesired ingestion, the level of stimulation may be increased until cessation is reached.

In some embodiments, patient management data is integrated into the treatment plan. Patient management data may be collected and recorded by the gastric stimulator, either alone or in combination with gastric stimulation treatment. Such patient management data includes data related to activity levels, sleep patterns, eating patterns, caloric intake, etc. Since such data is recorded by the stimulation system, false reporting by the patient in a diary or log is avoided. Patient management data may be recorded prior to treatment with gastric stimulation so that such data may be used in formulation of an initial treatment plan. Or patient management data may be recorded during treatment to monitor the patient and track improvement.

In a first aspect of the present invention, a system is provided for use in providing gastric stimulation to a patient, wherein the system includes an ingestion sensor, a stimulator, and a processor coupled to the sensor and the stimulator. The processor is configured to determine an ingestion of material by the patient, a desirability of the ingestion by the patient, and a level of stimulation based on the determination of ingestion and the determination of desirability of ingestion. The processor then induces the stimulator to transmit the level of stimulation. In many embodiments, the ingestion sensor comprises a temperature sensor, however a variety of sensors may be used.

In some embodiments, the processor comprises a module for determining the level of stimulation, wherein the module for determining the level of stimulation selects a level of no stimulation in response to a determination that material has been ingested by the patient and a determination that ingestion by the patient is desirable. Optionally, the module for determining the level of stimulation selects a level of stimulation below a personal threshold for the patient in response to a determination that material has been ingested by the patient and a determination that ingestion by the patient is desirable. Or, in some instances, the module for determining the level of stimulation selects a level of stimulation at or above a personal threshold for the patient in response to a determination that material has been ingested by the patient and a determination that ingestion by the patient is undesirable. In such instances, the module for determining the level of stimulation may include code for increasing the level of stimulation until a desired response is given by the patient.

In some embodiments, the processor comprises a module for determining the desirability of ingestion by the patient that includes a module for determining if ingestion occurs during a meal window. Optionally, the system may further comprise a real time clock and such a real time clock may be adjustable by a global positioning system.

In some embodiments, the processor comprises a module for determining the desirability of ingestion by the patient that includes a module for determining whether the material has a desirable compositional property. In some instances, the sensor comprises a compositional sensor configured to sense the compositional property of the ingested material.

In some embodiments, the processor comprises a module for determining the desirability of ingestion by the patient that includes a module for determining if the patient has a desirable activity level. In such instances the system may further comprise a motion sensor configured to sense motion of the patient or sense position of the patient.

In some embodiments, the processor comprises a module for determining the desirability of ingestion by the patient that includes a module for determining if the duration of the meal is acceptable. In other embodiments, the processor comprises a module for determining the desirability of ingestion by the patient that includes a module for determining if the patient is sufficiently hungry. In such embodiments, the system may further include a pH sensor, pressure sensor, mechanical sensor, or a biochemical sensor.

In a second aspect of the present invention, a system is provided for use in providing gastric stimulation to a patient, the system comprising a stimulator, and a processor coupled to the stimulator. The processor is configured to determine if current time is within a meal window, and a level of stimulation based on the determination of whether the current time is within the meal window, the level of stimulation being below a stop eating threshold for the patient in response to a determination that the current time is within the meal window. The processor then induces the stimulator to transmit the level of stimulation.

In some embodiments, the system further comprises a real time clock configured to provide current time and the real-time clock may be adjustable by a global positioning system. Optionally, the processor may comprise a module for determining if current time is within a meal window, wherein this module compares the current time to a predetermined meal time schedule.

In some embodiments, the processor comprises a module for determining the level of stimulation, wherein the module for determining the level of stimulation selects a level of stimulation below the stop eating threshold for the patient in response to a determination that ingested material has a desirable compositional property and that the current time is within the meal window.

In another aspect of the present invention, a system is provided for use in providing gastric stimulation to a patient, the system comprising a compositional sensor configured to sense a compositional property of ingested material, a stimulator, and a processor coupled to the stimulator. The processor is configured to determine desirability of the compositional property of the ingested material, and a level of stimulation based on the determination of the desirability of the compositional property. The processor then induces the stimulator to transmit the level of stimulation.

In some embodiments, the processor comprises a module for determining the level of stimulation, wherein the module selects a level of stimulation at or above a stop eating threshold for the patient in response to a determination that the ingested material has an undesirable compositional property.

In yet another aspect of the present invention, a system is provided for use in providing gastric stimulation to a patient, the system comprising a processor and a memory coupled to the processor, the memory configured to store a plurality of code modules for execution by the processor. The plurality of code modules comprises a module for determining if material has been ingested by the patient, a module for determining desirability of ingestion by the patient, and a module for determining a level of stimulation based on the determination of ingestion and the determination of desirability of ingestion.

In still another aspect of the present invention, a method is provided for gastric stimulation of a patient, the method comprising determining if material has been ingested by the patient, determining desirability of the ingestion by the patient, determining a level of stimulation based on the determined ingestion and the determined desirability of ingestion, and applying the determined level of stimulation to the patient from a stimulator implanted in the patient.

### BRIEF DESCRIPTION OF THE DRAWINGS

FIG. 1 illustrates an embodiment of a stimulation system of the present invention.

FIG. 2 illustrates another embodiment of a stimulation system of the present invention.

FIG. 3 illustrates a flowchart depicting an example stimulation protocol.

FIGS. 4-5 illustrate example sensor information obtained from a temperature sensor within a stomach of a patient.

FIG. 6 provides an example flowchart illustrating steps involved in determining if material has been ingested.

FIG. 7 illustrates example temperature measurements stored in a buffer split into time periods.

FIG. 8 illustrates examples of additional determinations in the determination f whether ingestion is desirable.

FIG. 9 illustrates an example wherein the determination of whether ingestion is desirable comprises the determination of whether ingestion occurs within a meal window.

FIG. 10 illustrates an example stimulation protocol based on the flowchart of FIG. 3 using the determination of desirability of ingestion of FIG. 9.

FIG. 11 illustrates an example wherein the determination of whether ingestion is desirable comprises the determination of whether the ingested material has a desirable compositional property.

FIG. 12 illustrates an example wherein the determination of whether ingestion is desirable comprises the determination of whether the patient has a desirable activity level.

FIG. 13 illustrates an example wherein the determination of whether ingestion is desirable comprises the determination of whether the duration of the meal is acceptable.

FIG. 14 illustrates an example wherein the determination of whether ingestion is desirable comprises the determination of whether the patient is sufficiently hungry.

FIG. 15 illustrates an example wherein the determination of whether ingestion is desirable comprises the determination of whether ingestion occurs within a meal window and optionally the combination of the determination of whether the ingested material has a desirable compositional property.

FIG. 16 illustrates an example wherein the determination of whether ingestion is desirable comprises the determination of whether the duration of the meal is acceptable and optionally the combination of determination of whether the ingested material has a desirable compositional property.

FIG. 17 illustrates an example wherein the determination of whether ingestion is desirable comprises the determination of whether the patient has a desirable activity level and optionally the combination of determination of whether ingestion occurred within a meal window.

FIG. 18 illustrates an example wherein the determination of whether ingestion is desirable comprises a determination of whether ingestion occurs within a meal window and optionally the combination of determination of whether the patient is sufficiently hungry.

FIG. 19 illustrates an example wherein the determination of whether ingestion is desirable comprises the determination of whether the patient is sufficiently hungry and the optional combination of the determination of whether the material has a desirable compositional property and further the optional combination of the determination of whether the patient has a desirable activity level.

FIG. 20 illustrates another example of a complex combination of determinations to determine desirability of ingestion.

FIG. 21 illustrates an embodiment wherein the determination of the level of stimulation is based on the determination of whether the current time is within a meal window.

FIG. 22 illustrates an embodiment wherein the determination of the level of stimulation is based on the determination of whether the current time is within a meal window and optionally on the determination of whether the material consumed has a desirable compositional property.

FIG. 23 illustrates an embodiment wherein the determination of the level of stimulation is based on the compositional desirability of ingested material.

### DETAILED DESCRIPTION OF THE INVENTION

In most instances, patients suffering from obesity have diminished ability to self-manage their daily food intake. Patients often overeat, snack between meals and generally make poor food choices. Methods, systems and devices are provided for using gastric stimulation to assist management of food intake. Such assistance may eventually cause learning of behavioral patterns by the patient, leading to corrected self-management.

Patient management for obesity treatment may be established according to the following goals and objectives:
Reducing total quantity of food consumed during meals
Reducing ingestion of food between meals
Reducing binging of food
Reducing intake of unhealthy foods
Reducing eating when not hungry
Increasing physical activity
Improving sleeping patterns, such as duration and restfulness (deep sleep)
   Each of these goals and objectives may be achieved with the use of gastric stimulation of the present invention in combination with various sensors.

Gastric stimulation may be achieved with the use of a variety of gastric stimulators, such as described in U.S. patent application Ser. Nos. 09/847,884 (U.S. Pat. No. 6,535,764), Ser. Nos. 10/109,296, 10/116,481, 10/691,880 (U.S. Pat. No. 7,020,531), Ser. Nos. 10/295,128, 10/290,788 (U.S. Pat. No. 7,016,735), Ser. Nos. 10/291,449, 10/295,115, 10/950,345, 10/888,218, 10/888,622, 10/992,382, 10/991,648, 11/256,264, 11/249,661, 11/249,290, 11/249,291, 11/281,234, 11/281,049, 60/815,640, each of which are incorporated herein by reference for all purposes. In some embodiments, the stimulator comprises electronic circuitry, optionally enclosed in a housing which may be implanted subcutaneously or attached to the stomach wall, and electronic leads that are coupled to the electronics circuitry. The leads include stimulating electrodes that are electrically couplable to the stomach wall. In some embodiments, the electronic circuitry includes a processor and a memory device having one or more code modules. The processor executes the one or more code modules to determine the level, duration and pattern of the stimulation. Typically, the electronic circuitry of the stimulator also includes a telemetry circuit for communication with separate devices, of which one may be for programming the stimulator's various operational parameters. It may be appreciated that memory may alternatively or additionally be located on the separate device.

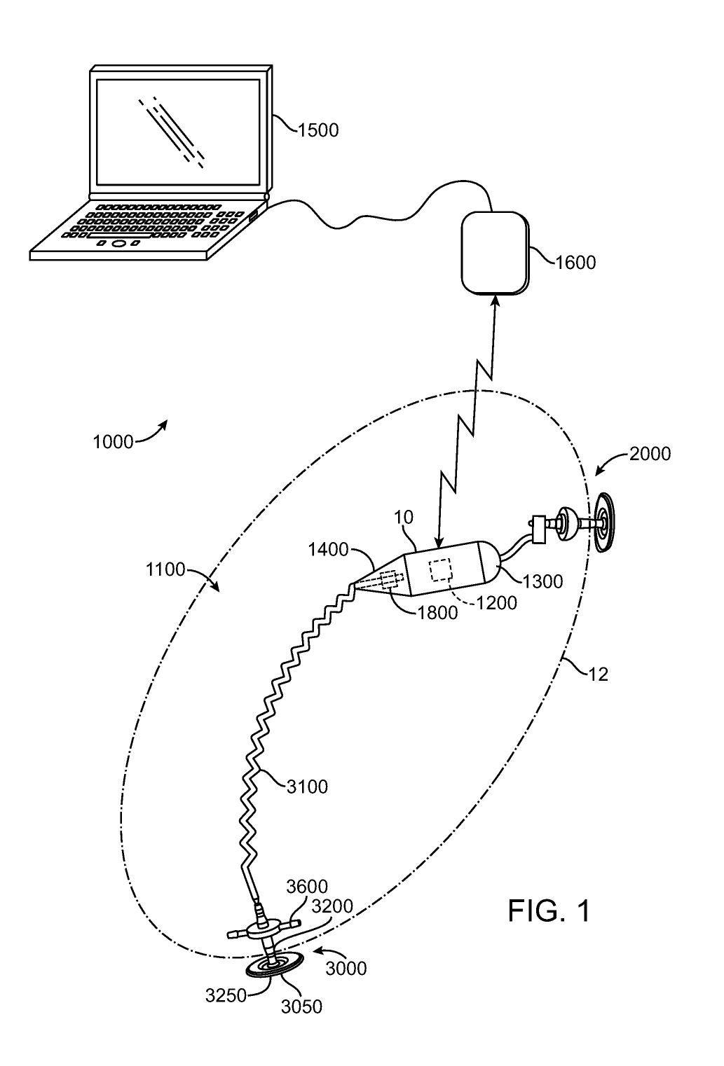

An example stimulation system 1000 is illustrated in FIG. 1. In this embodiment, the system 1000 comprises a stimulator 1100 which is implantable within an organ such as a stomach 12, small intestine or colon. The stimulator 1100 comprises implantable electronic circuitry 1200 contained within an implantable pulse generator (IPG) 10 which typically has a protective housing 1300. The housing 1300 is constructed of a corrosion resistant material, such as a material able to withstand implantation within a gastric environment. An IPG anchor 2000 is coupled to the IPG 10 and is configured to anchor the IPG 10 to a wall of the stomach. The stimulator 1100 also includes an electrode lead anchor 3000 comprising a first electrode 3200 and a return electrode 3250. The electrodes 3200, 3250 are coupled to the electronic circuitry 1200 through a flexible lead portion 3100 to a connector 1800 within header 1400 of housing 1300. The electrode lead anchor 3000 is configured to anchor the electrode 3200 so that it is in electrical contact with, or in proximity to the stomach wall 12. The electronic circuitry 1200 is configured to provide an electrically stimulating signal to a stomach wall via the electrodes 3200, 3250. While the electrodes 3200, 3250 are shown in particular configurations and locations, numerous electrode configurations and positions are contemplated. An external computer or programmer 1500 may be used to program various stimulation parameters or other instructions into a memory device included with the electronic circuitry 1200. The external programmer 1500 may be coupled to a telemetry device 1600 that communicates with the electronic circuitry via radio frequency signals.

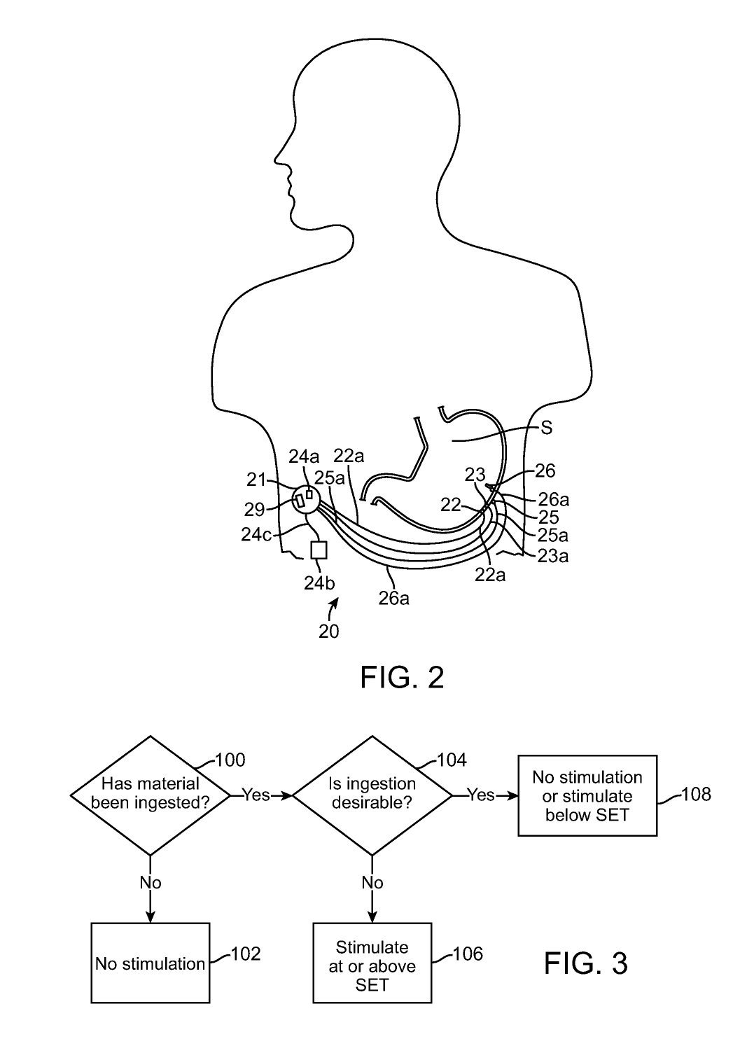

FIG. 2 illustrates another example of a stimulation system. This embodiment includes a stimulator 20 having an implantable pulse generator (IPG) 21 implanted subcutaneously within a patient. The stimulator further comprises leads 22a, 23a extending from the IPG 21 through the abdomen and to the stomach S where electrodes 22, 23 are implanted into the stomach muscle layer from the outside of the stomach S. The IPG 21 further comprises a sensor 24a located on the IPG 21 and/or a sensor 24b may be separate from the IPG and located elsewhere in the patient and coupled to the electronic circuitry 29 in the IPG by lead 24c. The stimulator also includes sensors 25, 26, that are implanted on or in the stomach S, respectively, with leads 25a, 26a extending from the sensors 25, 26 to the IPG 21. Sensor 26 is exposed to the inside of the stomach S while sensor 25 is attached to the outside of the stomach. Leads 22a, 23a, 24c, 25a, 26a are electrically coupled to the electronic circuitry 29 located in the IPG 21.

The gastric stimulators include or are used with at least one sensor for sensing information. The sensors may be located on or extend from the IPG and/or the sensors may be located on or extend from a lead or other device. Alternatively or additionally, a sensor may be located separately on the stomach wall and/or a sensor may be otherwise positioned elsewhere within, coupled to or in communication with the patient. The sensors and other responsive elements may include but are not limited to a number of types of sensors and responsive elements and any combination thereof. When the sensors are implanted in the stomach, they may sense ingestion of material, presence of material in the stomach, composition of such material, temperature, pH or pressure within the stomach, and/or patient motion corresponding to respiration or gross movement. Sensors positioned on the stomach may also sense various parameters that indicate the actions of the stomach, e.g. movement, contractions. The sensors may also utilize various imaging techniques (e.g. ultrasound or spectroscopy (absorption of various wavelengths of light) to identify ingestion composition of material in the stomach.

Example sensors include a temperature sensor, a pH sensor, an optical sensor, a pressure sensor, a mechanical/contraction sensor, a biochemical sensor, an alcohol sensor, a motion sensor/accelerometer, and an impedance sensor, to name a few. The stimulation device may be programmed to deliver stimulation in response to sensed parameters and/or the sensors may sense a plurality of parameters in order to determine whether or not to stimulate or otherwise respond. Alternatively or in addition, the stimulation device may be programmed to record sensor data without delivering stimulation. Thus, the device may be used to monitor the activities of the patient, such as eating patterns, activity levels, sleep duration and sleep quality, food quality, etc. In some embodiments, such monitoring is used for a period of time prior to treatment so that the patient's normal habits are accurately recorded for proper analysis and creation of an appropriate treatment protocol. The device may then be reprogrammed to delivery stimulation according to the treatment protocol and/or sensed parameters. In other embodiments, the stimulation device monitors certain activities of the patient and records such sensor data while simultaneously responding to certain sensed parameters. For example, the stimulation device may record continuous sensor data reflecting activity levels to provide an “exercise diary” while stimulating in response to sensed ingestion of food as such ingestion occurs. In such an example, the exercise diary may be retrieved at a later date for review while the ingestion patterns are temporal and not retrievable. It may be appreciated that any sensor data may be recorded and stored in combination with any other sensor data that is not recorded and stored. Overall, sensing may be used or over time to identify patterns, diagnose diseases and evaluate effectiveness of various treatment protocols.

In the embodiment of FIG. 1, circuitry 1200, telemetry device 1600, and external programmer 1500 are included in a data processing system of stimulation system 1000. Similarly, in the embodiment of FIG. 2, circuitry 29 may comprise a stand alone data processing system or may be configured to interface with one or more additional electronic components external of (and/or implanted at different locations within) the patient. Generally, the data processing systems included in embodiments of the invention may include at least one processor, which will typically include circuitry implanted in the patient, circuitry external of the patient, or both. When external processor circuitry is included in the data processing system, it may include one or more proprietary processor boards, and/or may make use of a general purpose desktop computer, notebook computer, handheld computer, or the like. The external processor may communicate with a number of peripheral devices (and/or other processors) via a bus subsystem, and these peripheral devices may include a data and/or programming storage subsystem or memory. The peripheral devices may also include one or more user interface input devices, user interface output devices, and a network interface subsystem to provide an interface with other processing systems and networks such as the Internet, an intranet, an Ethernet™, and/or the like. Implanted circuitry of the processor system may have some or all of the constituent components described above for external circuitry, with peripheral devices that provide user input, user output, and networking generally employing wireless communication capabilities, although hard-wired embodiments or other transcutaneous telemetry techniques could also be employed.

An external or implanted memory of the processor system will often be used to store, in a tangible storage media, machine readable instructions or programming in the form of a computer executable code embodying one or more of the methods described herein. The memory may also similarly store data for implementing one or more of these methods. The memory may, for example, include a random access memory (RAM) for storage of instructions and data during program execution, and/or a read only memory (ROM) in which fixed instructions are stored. Persistent (non-volatile) storage may be provided, and/or the memory may include a hard disk drive, a compact digital read only memory (CD-ROM) drive, an optical drive, DVD, CD-R, CD-RW, solid-state removable memory, and/or other fixed or removable media cartridges or disks. Some or all of the stored programming code may be altered after implantation and/or initial use of the device to alter functionality of the stimulator system.

The functions and methods described herein may be implemented with a wide variety of hardware, software, firmware, and/or the like. In many embodiments, the various functions will be implemented by modules, with each module comprising data processing hardware and/or software configured to perform the associated function. The modules may all be integrated together so that a single processor board runs a single integrated code, but will often be separated so that, for example, more than one processor board or chip or a series subroutines or codes are used. Similarly, a single functional module may be separated into separate subroutines or be run in part on separate processor chip that is integrated with another module. Hence, a wide variety of centralized or distributed data processing architectures and/or program code architectures may be employed within different embodiments.

The electronic circuitry comprises and/or is included within a controller or processor for controlling the operations of the device, including sensing, stimulating, signal transmission, charging and/or using energy from a battery device for powering the various components of the circuit, and the like. As such, the processor and battery device are coupled to each of the major components of the implanted circuit. In some embodiments, the electronic circuitry includes an internal clock. The internal clock may also include a real time clock component. The internal clock and/or real time clock may be used to control stimulation, e.g. by stimulating or allowing stimulation at a particular time of the day. The real time clock component may also provide a date/time stamp for detected events that are stored as information in a memory device. Optionally, the memory may be preserved by saving information corresponding to an event of interest which is saved along with the time/date when the event occurred.

The memory device is configured to store a plurality of code modules for execution by the processor. The code modules provide a variety of determinations based on sensor information and various other inputs, such as information from the internal clock, which are used to actuate a stimulation driver. The stimulation driver is coupled to the stimulating electrodes that are used to provide electrical stimulation.

Referring to FIG. 3, a flowchart depicting a stimulation protocol of the present invention is provided. Here, the stimulation protocol begins with the determination of whether material has been ingested by the patient (step 100). Such a determination may be based on signals from one or more sensors, input from the patient, or other mechanisms as will be discussed in later sections. The memory device of the stimulator also includes a module for making such determination. If no ingestion has been determined, no stimulation will be provided (step 102). Thus, the stimulator remains quiet when the patient is not eating. This conserves energy and ultimately battery life. If it is determined that ingestion has occurred, it is then determined whether such ingestion is desirable (step 104). Desirability of ingestion is based on one or more factors which will also be discussed in detail in later sections. The memory device also includes a module for making this determination of desirability. If the ingestion is determined to be undesirable, stimulation is provided at a level at or above a “stop eating threshold” SET (step 106) for the patient that typically causes the patient to feel a displeasurable sensation, such as gastric discomfort such as to the extent of nausea, pain or vomiting. Such a displeasurable sensation is one which causes the patient to stop the undesired ingestion. Since each patient may react differently to the same level of stimulation, the SET will be customized for each patient by prior testing of the patient's response to gastric stimulation. If the patient does not stop the undesired ingestion, the level of stimulation may be increased until cessation is reached.

In some embodiments, if the ingestion is determined to be desirable, no stimulation is provided (step 108). Thus, the patient is able to eat without stimulation with the assumption that such ingestion is allowed. In other embodiments, if ingestion is determined to be desirable, stimulation is provided at a level below the SET (step 108) for the patient. Stimulation below the SET may include a variety of gastric sensations, including bloating, salivation, fullness, dyspepsia and early satiety. The intent of stimulating below the SET while the patient is consuming is to decrease the overall quantity of ingested material. Patients feel full sooner, curtail eating time and typically eat less when stimulated below the SET during consumption. This will also be described in detail in a later section.

It may be appreciated that the actual physical sensations associated with different levels of stimulation may vary from patient to patient and from incident to incident for the same patient. Also, a particular sensation, such as nausea, may be felt at a SET by one patient and not by another. Therefore, the SET is determined by patient behavior, rather than elicited sensations, and is established for an individual patient during a preliminary testing period. During use, stimulation is provided at, above or below the SET depending on the sensed behavior of the patient. If the desired resultant behavior is not attained, such as immediate cessation of eating, the stimulation can then be increased at that time to achieve the desired result.

Thus, in some embodiments, the memory device includes a module for determining a level of stimulation based on the determination of ingestion and the determination of desirability of ingestion.

Determining if Material has been Ingested

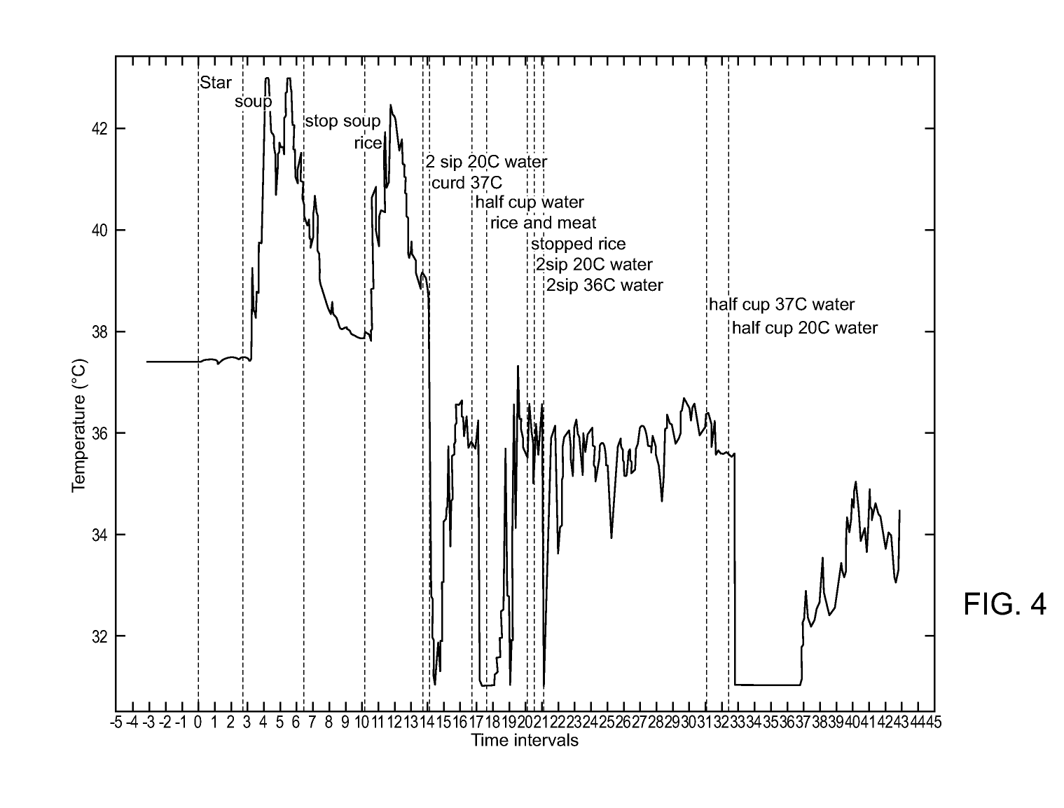

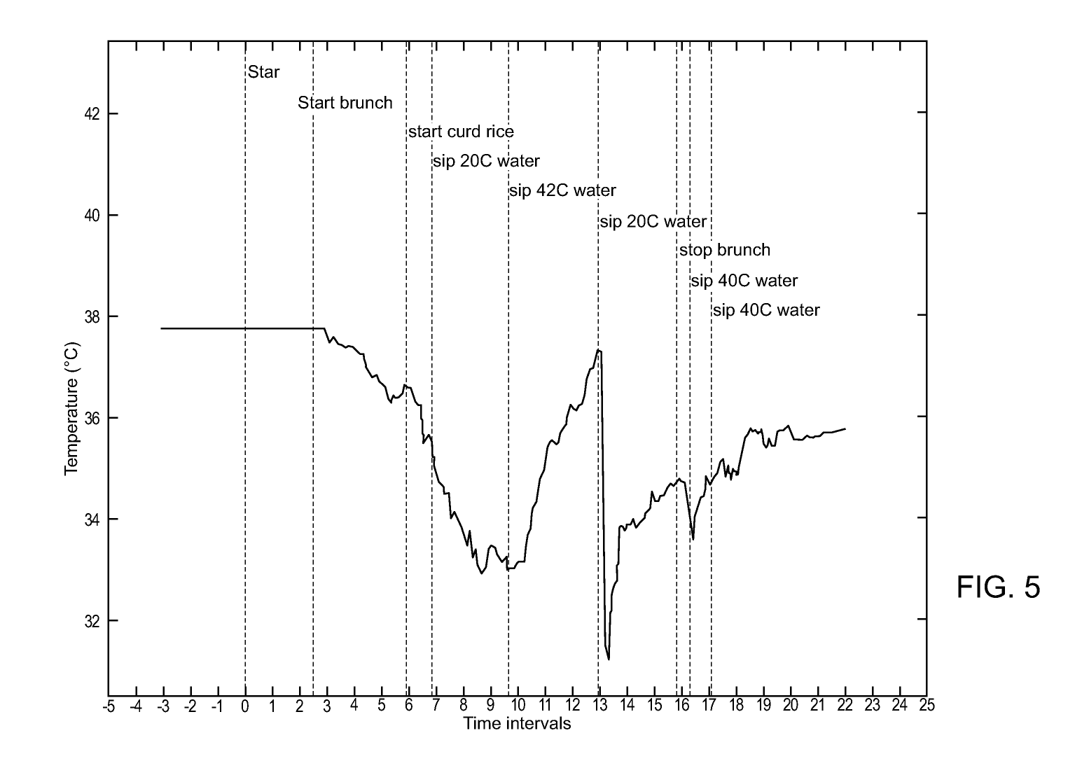

The determination of whether material has been ingested is based on sensor information from one or more ingestion sensors. In some embodiments, such sensor information is provided from a temperature sensor disposed at least partially within the stomach lumen so that temperature changes within the stomach can be sensed. For example, the ingestion of a hot beverage or meal item will immediately register an increase in temperature by the sensor as the sensor senses the presence of the hot ingested material. Likewise, ingestion of ice water or a cold meal item will register a decrease in temperature by the sensor. FIGS. 4-5 illustrate example sensor information obtained from a temperature sensor within a stomach of a patient during consumption of various foods. FIG. 4 illustrates temperatures detected while a patient consumes a meal. As shown, the patient begins by consuming a warm soup wherein a responsive temperature rise is shown. Throughout the meal, the patient eats food of various temperatures and drinks water of various temperatures with responsive changes in sensed temperature. Similarly, FIG. 5 illustrates temperatures detected while a patient consumes brunch. As shown, the patient eats food of various temperatures and drinks water of various temperatures with responsive changes in sensed temperature.

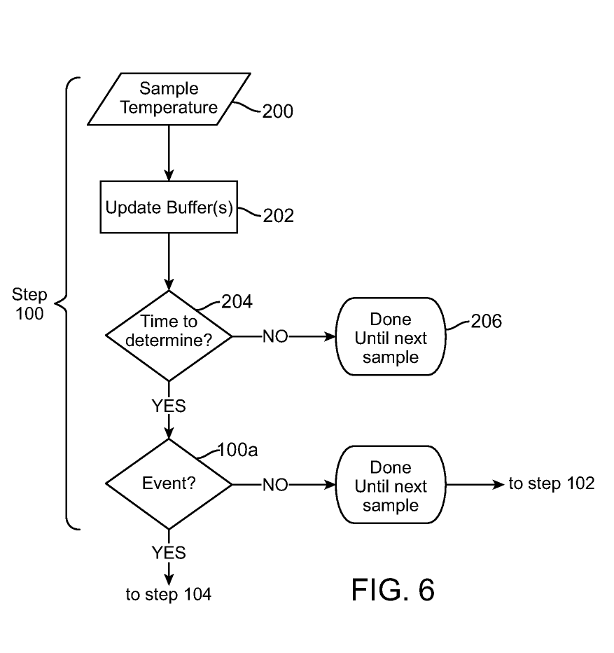

FIG. 6 provides an example flowchart illustrating steps involved in determining if material has been ingested (step 100). To begin, temperature is sampled at a predetermined rate (step 200). The sampling rate is high enough to capture fast transients yet conserves memory, processor time and power. In some embodiments, temperature is sampled at 6 second intervals. The temperature measurements are recorded to a buffer (step 202). The size of the buffer maximizes the accurate determination of ingestion of material yet conserves memory, computation requirements and resultant delay. In some embodiments, the buffer includes 32 samples, spanning the previous 3 minutes and 6 seconds. While temperature measurements are made every sampling period, determinations of ingestion are made less frequently. Thus, at each sampling time, a decision is made (step 204) as to whether it is time to determine if material has been ingested. If it is not time, then no action is taken until the next sample is acquired (step 206). If it is time, calculations are made to determine if an ingestion event (100a) has occurred.

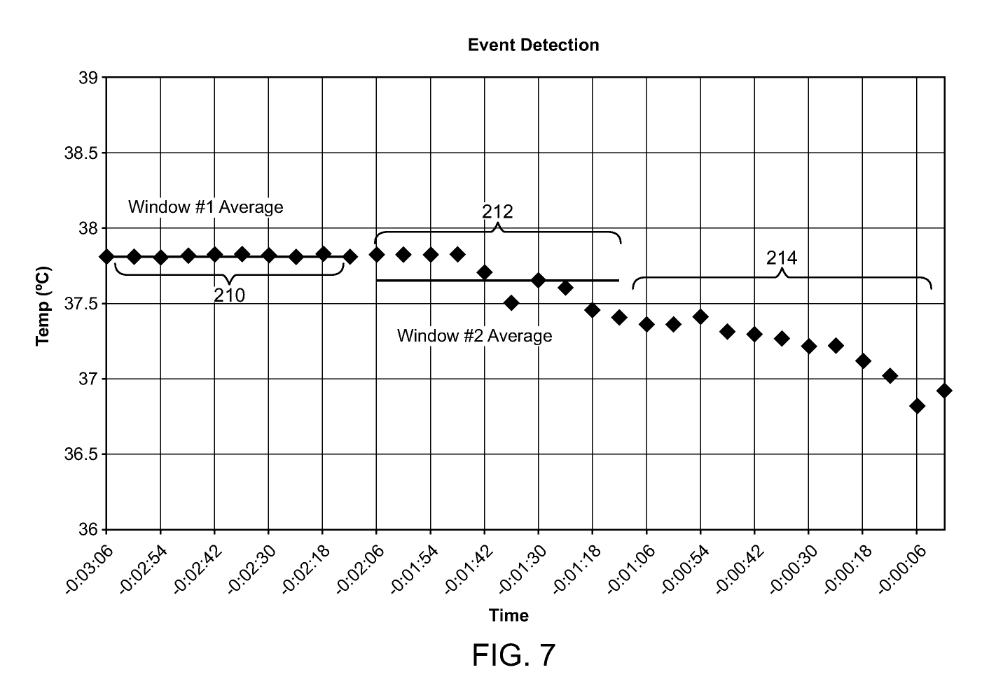

In some embodiments, the calculations include splitting the temperature measurements in the buffer into three time periods ranging from oldest to newest. FIG. 7 illustrates example temperature measurements stored in the buffer split into a first time period 210, a second time period 212 and a third time period 214. In this embodiment, the average of the temperature measurements of the oldest or first time period 210 are calculated and compared to the temperature measurements of the second time period 212. If the difference exceeds a predetermined threshold, it is determined that ingestion has occurred. Thus, referring to FIG. 6, the processor would then proceed to the next code module which determines if the ingestion is desirable (step 104). If the difference does not exceed a predetermined threshold, it is determined that ingestion has not occurred. In such an instance, no stimulation would occur (step 102).

In some embodiments, the buffer of temperature measurements is used to differentiate between eating and drinking. In such embodiments, if it has been determined that ingestion has occurred, the processor then executes a code module which determines if eating has occurred or if drinking has occurred. In some embodiments, stimulation response is based on whether the patient is eating or drinking. For example, the response may be more aggressive in relation to eating than drinking. Thus, patients may be encouraged to consume beverages, such as water.

In some embodiments, temperature changes due to the sensing of ingested material are differentiated from common body temperature changes with the use of a plurality of temperature sensors. In such embodiments, at least one sensor is disposed within the stomach to measure temperature within, and at least one sensor is disposed outside of the stomach. For example, when the stimulator has a housing implanted subcutaneously within a patient, the sensor may be disposed on or within the housing. Any common body temperature changes would occur in both sensors while temperature changes due to ingestion would only affect the sensor within the stomach. Thus, temperature changes due to ingestion may be differentiated from general body temperature fluctuations.

It may be appreciated that other ingestion sensors may be used. Example ingestion sensors include pH sensors, mechanical sensors, strain gauges, contraction sensors, electrical sensors, impedance sensors, pressure sensors, biochemical sensors, optical emitters and sensors, and the like. The ingestion sensors may be used alone, in plurality or in any combination.

### Determining if Ingestion is Desirable

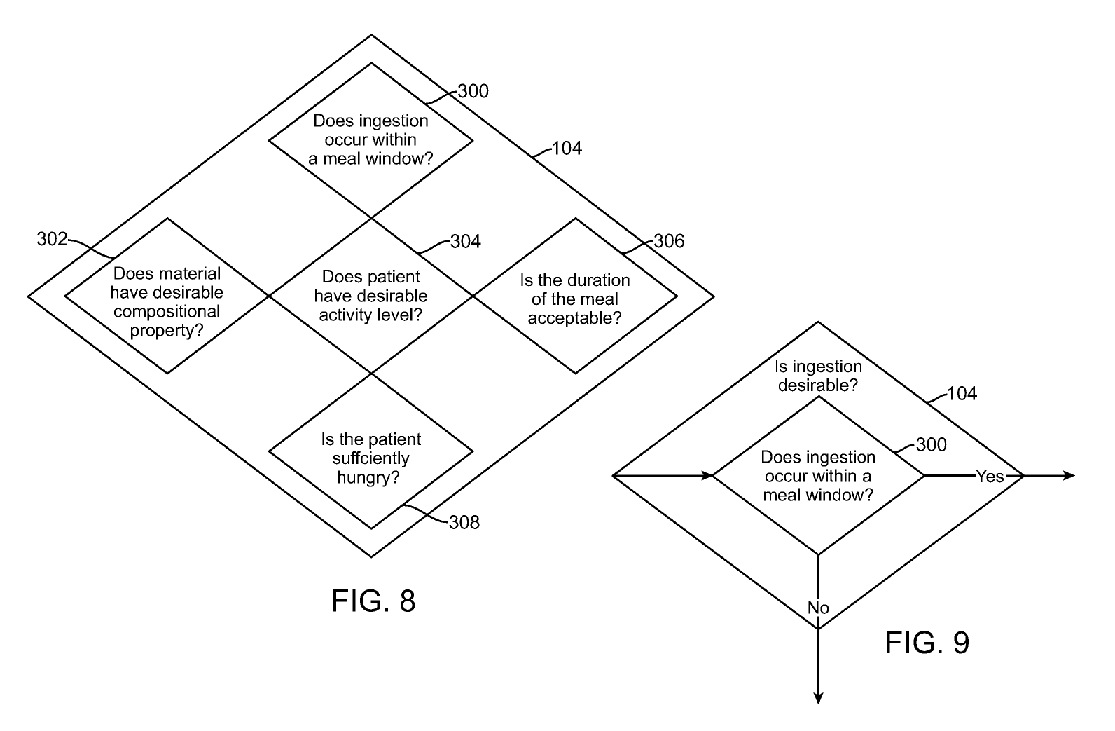

The determination of whether ingestion is desirable is based on one or more additional determinations. Examples of such additional determinations are illustrated in FIG. 8 and include:
Determining whether ingestion occurs within a meal window (step 300)
Determining whether ingested material has a desirable compositional characteristic (step 302)
Determining whether the patient has a desirable activity level (step 304)
Determining whether the duration of the meal is acceptable (step 306)
Determining whether the patient is sufficiently hungry (step 308)
   These determinations 300, 302, 304, 306, 308 can each be used to determine if ingestion by the patient is desirable at any given time. Likewise, any combination of these determinations, or any combination of any subset of these determinations, can also be used to ultimately determine if ingestion is desirable. Example combinations of these determinations will be illustrated in a later section. Each type of determination will be described in more detail herein:

### Meal Windows

FIG. 9 illustrates an example wherein the determination of whether ingestion is desirable (step 104) comprises the determination of whether ingestion occurs within a meal window (step 300). Meal windows may be predetermined for an individual patient to encourage eating during specific time periods and discourage eating outside of these time periods. For example, desired meal windows may include 8:00-8:30 am (breakfast), 12:00 pm-12:30 pm (lunch) and 6:00-6:30 pm (dinner). This may establish regular healthy eating patterns and diminish undesired habits, such as snacking between meals and late night eating. The predetermined meal time windows may also reduce binging or extending eating by providing a designated length of time within which the meal is to be consumed.

In these embodiments, the gastric stimulator includes a timer or internal clock, such as a real time clock. The clock may include time of day, day of week, date, year, or any combination, to name a few. The clock may have the capability of being set by the patient. However, to improve patient compliance, the clock may have a feature which restricts setting or resetting to specific individuals, such as with a code or key. Alternatively or in addition, the clock may be adjusted or calibrated to a specific time with the use of GPS or similar system. Such calibration may be useful during travel, such as crossing various time zones. The clock may also be used in creating a timestamp, e.g. recording the time in which an event occurred. Such an event may be a signal provided by a sensor, such as sensed ingestion. The timestamps may be stored in the memory device and used to record behavior of the patient. Thus, the gastric stimulator may used as a recorder to record the eating patterns of the patient prior to treatment. Such recording can be used to tailor the treatment protocol to the individual needs of the patient. The timestamps may also be used in the determinations by the processor, such as to determine the level of stimulation to provide at a given moment.

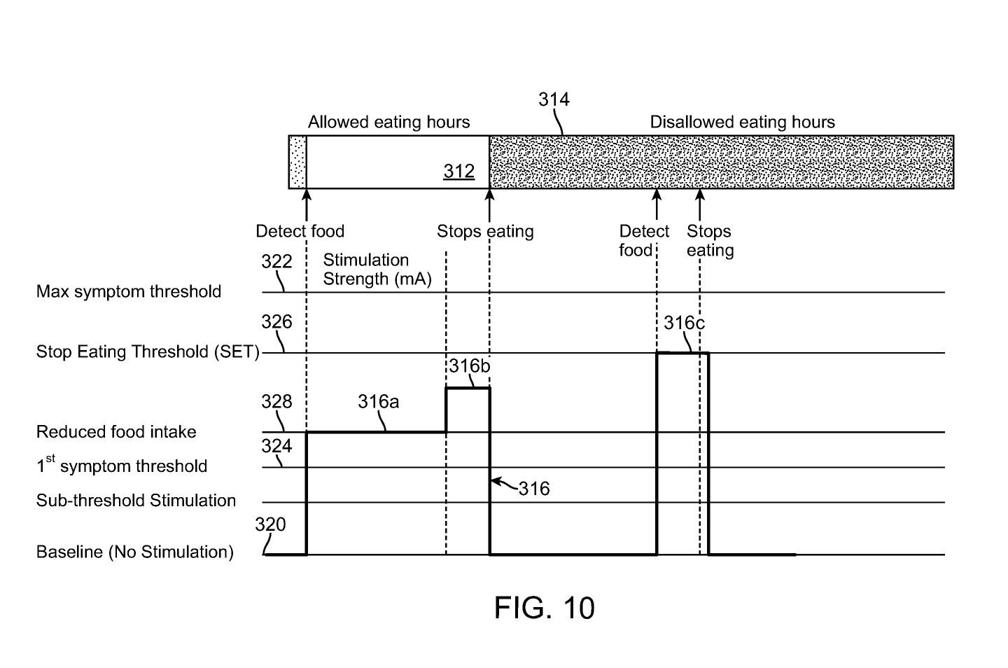

FIG. 10 illustrates an example stimulation protocol based on the flowchart of FIG. 3 using the determination of desirability of ingestion of FIG. 9. A meal window 312 is illustrated within a time frame 314. And, a stimulation strength or stimulation level curve 316 is illustrated in relation to the time frame 314 showing the changing levels of stimulation. The stimulation level may vary between baseline or no stimulation 320 and a maximum symptom threshold 322. Various threshold levels may be established for an individual patient during a preliminary testing period. Example thresholds include a first symptom threshold 324, which is determined based on the lowest stimulation which evokes a symptom such as gastric discomfort, and a SET 326, which is determined based on the stimulation which causes the patient to immediately stop eating. Between the first symptom threshold 324 and the SET 326 resides a level of stimulation which reduces intake 328 by the patient.

In the example of FIG. 10, the patient begins eating at the start of the meal window 312. The processor executes the module for determining the level of stimulation based on the positive determination of ingestion, the real time clock, and the consequent positive determination of desirability of ingestion. The stimulation strength increases to the reduced food intake level 328 as illustrated by the stimulation level curve 316a. This is maintained throughout the meal window 312, and in this example, is raised slightly 316b in anticipation of the end of the meal window 312 to further curtail eating. After the patient stops eating, the stimulation drops down to baseline. This can be sensor based or time based. If the ingestion is detected outside of the meal window 312, the stimulation strength increases to the SET 326 as illustrated by the stimulation level curve 316c. This causes the patient to immediately discontinue eating. It may be appreciated that immediateness may vary and is considered to be significantly shorter than a typical meal. Also, stimulation may be increased above the SET 326 to assist in the discontinuance of eating. Once ingestion is no longer sensed, the stimulation returns to baseline.

### Compositional Properties

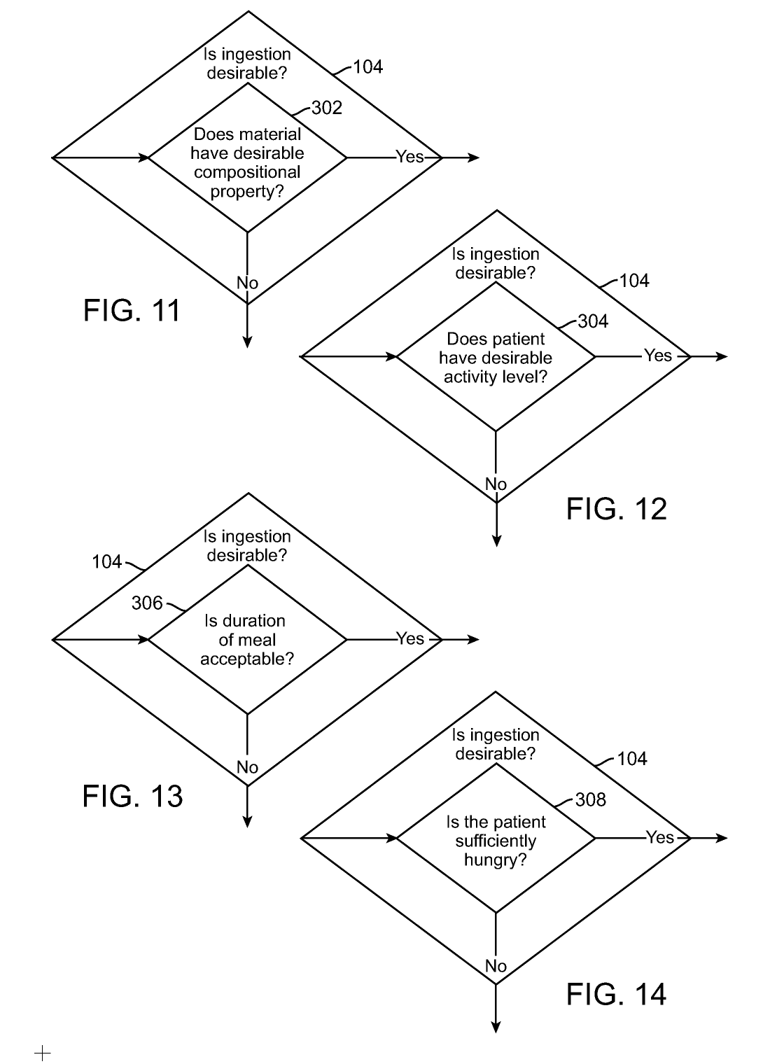

FIG. 11 illustrates an example wherein the determination of whether ingestion is desirable (step 104) comprises the determination of whether the ingested material has a desirable compositional property (step 302). A desirable compositional property is a property which is considered healthful or dietarily acceptable for a given patient. Most foods are compositionally complex materials made up of a variety of different chemical constituents. Their composition can be specified according to a variety of properties, such as specific atoms (e.g. carbon, hydrogen, oxygen, nitrogen, sodium, etc.), specific molecules (e.g. water, sucrose, tristearin, etc.), types of molecules, (e.g. fats, proteins, carbohydrates, fiber, minerals, etc.) or specific substances (e.g. peas, flour, milk, butter, peanuts, etc). In some embodiments, the present invention includes mechanisms and devices which identify one or more such properties of the ingested material. The mechanisms can be tailored to identify any of the above described properties depending on how the treatment protocol is designed. For example, a patient may be restricted from eating foods having a fat content over a predetermined amount. Or, a patient may be restricted from eating particular foods, such as butter, which are considered unhealthy. Or the patient may be allowed to consume beverages, such as water, which are considered to be healthy. In some instances, the patient may be allowed to consume low calorie artificial sweeteners but not sugar. Each of these properties of the ingested material may be determined and utilized in the determination of whether the ingestion is desirable. The consequent stimulation is then provided to the patient.

A variety of mechanisms and devices may be used for identifying such compositional properties and may be considered compositional sensors. In many embodiments, such mechanisms utilize spectroscopy, e.g. UV-visible, fluorescence, atomic, infrared, near-infrared (NIR) and nuclear magnetic resonance spectroscopes. Such mechanisms utilize interactions between electromagnetic radiation and matter. The type of mechanism based on spectroscopy depends on the nature of the energetic transitions involved, (e.g. electronic, vibration, rotation, translation, nuclear), the nature of the radiative process involved (e.g. absorption, emission, fluorescence) and the nature of the food matrix (e.g. absorbing, non-absorbing). These factors determine the wavelength or frequency of electromagnetic radiation used, the way that the electromagnetic radiation is generated and the way that the electromagnetic radiation is detected.

Thus, a variety of spectroscopic analyses and other mechanisms may be utilized in the present invention. Such mechanisms include known analytical procedures for characterizing food samples. Example procedures are used in major sectors of the food industry, including food manufacturers, ingredient suppliers, analytical service laboratories, government agencies (FDA, USDA, etc), and University research laboratories. For example, NIR spectroscopy is used routinely for the compositional, functional and sensory analysis of food ingredients, process intermediates and final products (“Near-infrared Spectroscopy in Food Analysis”, Encyclopedia of Analytical Chemistry, ed. Robert A. Meyers, John Wiley & Sons Ltd. ISBN 0471976709, incorporated herein by reference for all purposes).

In some embodiments, the determination of whether the ingested material has a desirable compositional property is based on the presence of one or more markers. Markers may be incorporated into prepackaged or prepared food that is designated for the patient to consume according to the treatment protocol. For example, a variety of current dietary programs include prepared meals, such as Jenny Craig®, Weight Watchers®, etc. The patient is instructed to consume the meals provided by the program according to a schedule in order to control the food quality and quantity that the patient eats. However, such programs do not prevent the patient from eating foods outside of the program and therefore rely on the discipline of the patient alone for success. The present invention provides markers within the food of the prepared meals and the markers are detected by sensors or other devices in communication with the gastric stimulator. If the ingested material does not include a detectable marker, the material is determined to not have a desirable compositional property and stimulation is delivered at or above the SET. If the ingested material does include a detectable marker, the material is determined to have a desirable compositional property and no stimulation is delivered or stimulation below the SET is delivered.

A variety of markers may be used. Example markers include any biocompatible markers, such as fluorescent markers, that are food-safe. Other types of markers include food-safe quantum dots, such as related to a Type 2 EviTag™ luminescent label (Evident Technologies; Troy, N.Y.).

### Activity Level

FIG. 12 illustrates an example wherein the determination of whether ingestion is desirable (step 104) comprises the determination of whether the patient has a desirable activity level (step 304). In some embodiments, the patient is encouraged to increase activity levels to burn calories, build muscle tone, improve overall health, etc. During such activity, the patient may desire to consume liquids, such as water or a sports drink, or solid food, such as an energy bar. To assist in such encouragement and to ensure adequate hydration and sustenance for the patient during such exercise, the stimulation level may be determined based on the activity level of the patient. The activity level of the patient may be determined with the use of one or more of a variety of sensors, including an accelerometer.

In some embodiments, a 3-axis MEMS-type accelerometer is used. This accelerometer provides a voltage offset on each of the 3 axes, which can be used to determine position of the accelerometer, and, after calibration, position of the patient (e.g. lying down or standing upright). This accelerometer also provides an increased voltage from the offset based on motion. The level of this voltage can be used as an indication of the activity level of the patient (i.e. the voltage will be greater as the activity level increases).

In other embodiments, the activity level of the patient is determined using a 1- or 2-axis accelerometer, or a piezo sensor. Examples of such are those currently used in conventional pacemakers and defibrillators.

The activity sensor can also be used to monitor sleeping patterns, such as duration and restfulness. It has been found that some patients overeat to compensate for lack of sleep. Thus, sleep duration can be recorded with the use of the stimulation device and such information can be used in treatment of the patient. Alternatively, the stimulation device may be automatically shut off during periods of sensed sleep so as to conserve battery life.

### Duration of Meal

FIG. 13 illustrates an example wherein the determination of whether ingestion is desirable (step 104) comprises the determination of whether the duration of the meal is acceptable (step 306). Limiting the duration of the meal may assist in reducing episodes of binging by the patient. Thus, a limit on the meal time may be set at, for example, 20 minutes after the commencement of the meal. Such limitations differ from meal windows in that the limit or “end time” for the meal is not based on the time of day or a predetermined sequence of meal times, but rather on duration of time since the commencement of the meal. Therefore, such binge control may be applied to patients who do not desire the restriction of eating at specific times of day but may benefit from meal time limitations. Such a feature essentially creates “moving meal windows”—a meal window created when the patient decides to ingest food.

Once ingestion has been detected, the event is time-stamped and stored by the memory device. This event begins the meal which is allowed a predetermined duration time. When ingestion of material is detected thereafter, the time elapsed since the commencement of the meal is compared to the predetermined duration time. If the time elapsed is less than the predetermined duration time, the duration of the meal is considered acceptable, and therefore the ingestion is considered desirable. No stimulation or stimulation below the SET ensues, per the sequence outlined in FIG. 3. Once the time elapsed exceeds the predetermined duration time, the duration of the meal is considered unacceptable, and therefore ingestion is considered undesirable. Stimulation at or above the SET ensues, per the sequence outlined in FIG. 3.

To ensure that the patient is not consuming meals back to back, each ingestion event may be time-stamped and stored by the memory device. The pattern of ingestion events is then used to determine which ingestion event marks the commencement of a meal.

In some embodiments, the commencement of a meal is indicated by the patient. The patient is given an activator that is positionable near or against the body. The patient presses a button on the activator, or similarly activates a switch, that triggers by telemetry the stimulation device to time stamp the event. In these embodiments, the patient may be instructed that, for example, four meals are allowed in a 24 hour period. They can use their meals at any time, however additional meals will not be allowed. Each of the meals are limited by the predetermined duration time and eating between meals is considered undesirable. Therefore, patients will be motivated to register the commencement of a meal to allow themselves a meal time. They will also be unmotivated to register too many meals back to back since, in this example, they know they are only allowed four meals per day. Such a system would be ideal for patients who have gained some level of self-regulation, such as through use of the gastric stimulator of the present invention, and can handle increased control over meal times but would still like assistance from the device.

### Hunger Level

FIG. 14 illustrates an example wherein the determination of whether ingestion is desirable (step 104) comprises the determination of whether the patient is sufficiently hungry (step 308). Many patients eat for various reasons other than hunger, such habit, boredom, stress, anxiety, etc. Thus, patients tend to eat more often than they are hungry which can lead to weight gain. In addition, associations between these emotions and eating are formed which continues the pattern leading to continual weight gain. To break this cycle and retrain the patient to reduce eating when not sufficiently hungry, gastric stimulation may be governed at least in part by the level of patient hunger.

The level of patient hunger may be sensed by one or more sensors such as by a pH sensor, pressure sensor, mechanical/contraction sensor, or a biochemical sensor such as a leptin or ghrelin sensor, to name a few. In some embodiments, a blood glucose sensor is used. In other embodiments, acid secretion levels are sensed. In yet other embodiments, the start of slow waves that correlate with hunger are sensed.

In this embodiment, desirability of ingestion is dependent upon whether the patient is sufficiently hungry. When ingestion is detected, the processor executes the module for determining if the patient is sufficiently hungry which in turn determines if the ingestion is desirable. The processor then executes the module for determining the level of stimulation based on the positive determination of ingestion and the determination of desirability of ingestion, as illustrated in FIG. 3.

### Combinations

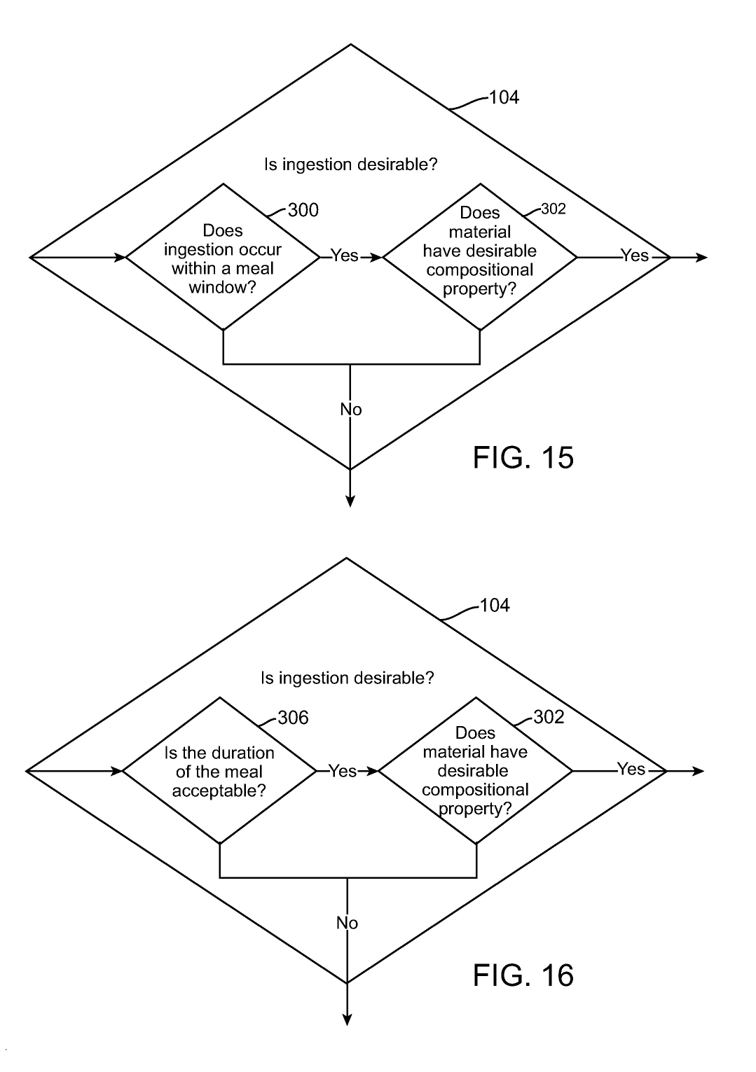

As mentioned previously, the above described determinations 300, 302, 304, 306, 308, or any subset of these determinations, can be combined in any arrangement to ultimately determine if ingestion is desirable. FIGS. 15-20 illustrate some example combinations of these determinations. Such examples are illustrative and not considered to be limiting in scope of the present invention.

FIG. 15 illustrates an example wherein the determination of whether ingestion is desirable (step 104) comprises the determination of whether ingestion occurs within a meal window (step 300) and optionally the combination of the determination of whether the ingested material has a desirable compositional property (step 302). After it has been positively determined that material has been ingested (step 100), the processor then executes the module to determine whether the ingestion occurs within a meal window (step 300). If this is a negative determination (i.e. the patient is consuming outside of a meal window), the ingestion is considered undesirable and the patient is provided stimulation at or above the SET (step 106), per FIG. 3. Thus, any eating outside of the meal window, regardless of the composition of the food, is restricted.

If there is a positive determination (i.e. the patient is consuming within the meal window (step 300)), the processor then executes the module to determine if the material has a desirable compositional property (step 302). If so, the ingestion is considered desirable and the patient is provided with no stimulation or stimulation below the SET (step 108) per FIG. 3. If not, the ingestion is considered undesirable and the patient is provided stimulation at or above the SET (step 106), per FIG. 3. Thus, the patient must eat during a meal window and must eat food that is acceptable (e.g. healthy, prescribed, allowable, etc) to avoid stimulation at or above the SET.

FIG. 16 illustrates an example wherein the determination of whether ingestion is desirable (step 104) comprises the determination of whether the duration of the meal is acceptable (step 306) and optionally the combination of determination of whether the ingested material has a desirable compositional property (step 302). After it has been positively determined that material has been ingested (step 100), the processor then executes the module to determine whether the duration of the meal is acceptable (step 306). If this is a negative determination (i.e. the patient is binging or consuming beyond the predetermined duration of time), the ingestion is considered undesirable and the patient is provided stimulation at or above the SET (step 106), per FIG. 3. Thus, any extended eating, regardless of the composition of the ingested material, is deterred.

If there is a positive determination (i.e. the patient is consuming within the acceptable duration of time (306)), the processor then executes the module to determine if the material has a desirable compositional property (step 302). If so, the ingestion is considered desirable and the patient is provided with no stimulation or stimulation below the set (step 108) per FIG. 3. If not, the ingestion is considered undesirable and the patient is provided stimulation at or above the SET (step 106), per FIG. 3. Thus, the patient must eat within the acceptable duration of time and must eat food that is acceptable (e.g. healthy, prescribed, allowable, etc) to avoid stimulation at or above the SET.

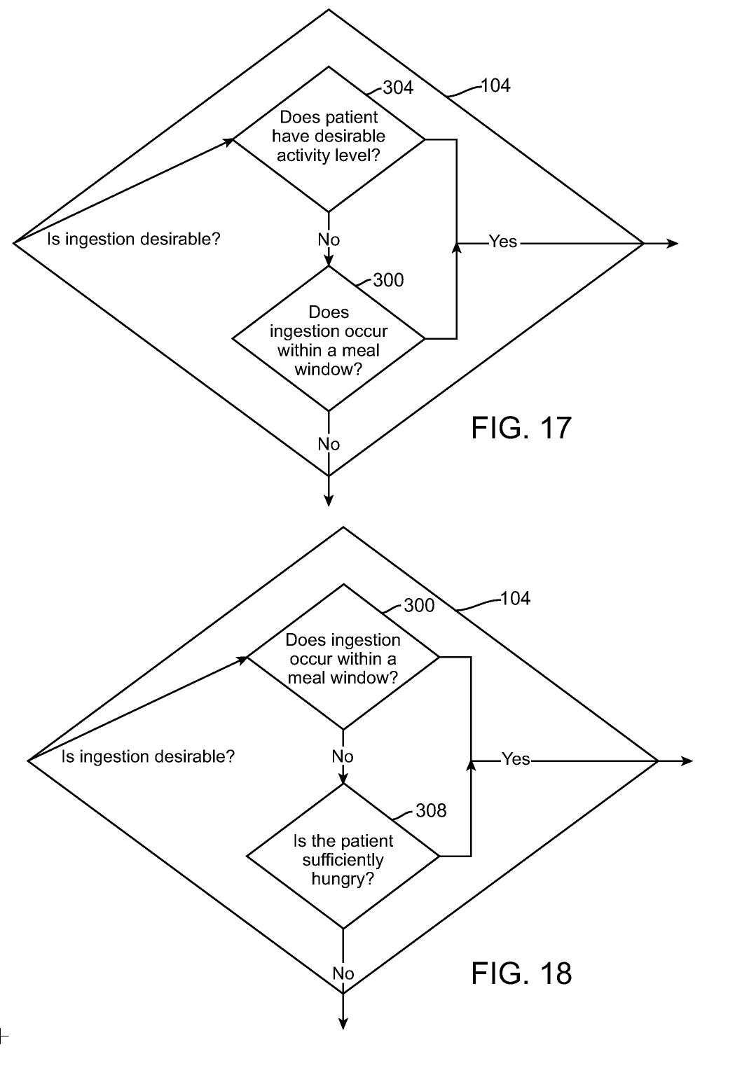

FIG. 17 illustrates an example wherein the determination of whether ingestion is desirable (step 104) comprises the determination of whether the patient has a desirable activity level (step 304) and optionally the combination of determination of whether ingestion occurred within a meal window (step 300). After it has been positively determined that material has been ingested (step 100), the processor then executes the module to determine whether the patient activity level is desirable (step 304). If this is a positive determination (e.g. the patient is exercising), the ingestion is considered desirable and the patient is provided with no stimulation or stimulation below the SET (step 108) per FIG. 3. Thus, any consumption during exercise is allowed.

If there is a negative determination (e.g. the patient is not exercising or sustaining a high enough level of activity), the processor then executes the module to determine if the ingestion is occurring within a meal window (step 300). If so, the ingestion is considered desirable and the patient is provided with no stimulation or stimulation below the set (step 108) per FIG. 3. If not, the ingestion is considered undesirable and the patient is provided stimulation at or above the SET (step 106), per FIG. 3. Thus, the patient may ingest at any time while maintaining a desirable activity level but is otherwise restricted to ingestion during meal windows. This allows the patient to readily consume water, sports drinks or other sustenance while exercising. This may also motivate the patient to exercise more.

FIG. 18 illustrates an example wherein the determination of whether ingestion is desirable (step 104) comprises a determination of whether ingestion occurs within a meal window (step 300) and optionally the combination of determination of whether the patient is sufficiently hungry (308). After it has been positively determined that material has been ingested (step 100), the processor then executes the module to determine whether the ingestion occurs within a meal window (step 300). If this is a positive determination, (i.e. the patient is consuming within a meal window), the ingestion is considered desirable regardless of actual hunger levels. If this is a negative determination (i.e. the patient is consuming outside of a meal window), it is then determined whether the patient is sufficiently hungry (step 308). If so, the ingestion is considered desirable and the patient is provided with no stimulation or stimulation below the SET (step 108) per FIG. 3. If not, the ingestion is considered undesirable and the patient is provided stimulation at or above the SET (step 106), per FIG. 3. Thus, the patient is not deterred from eating outside of meal windows if sufficiently hungry. However, emotional eating or other non-hunger related eating is deterred.

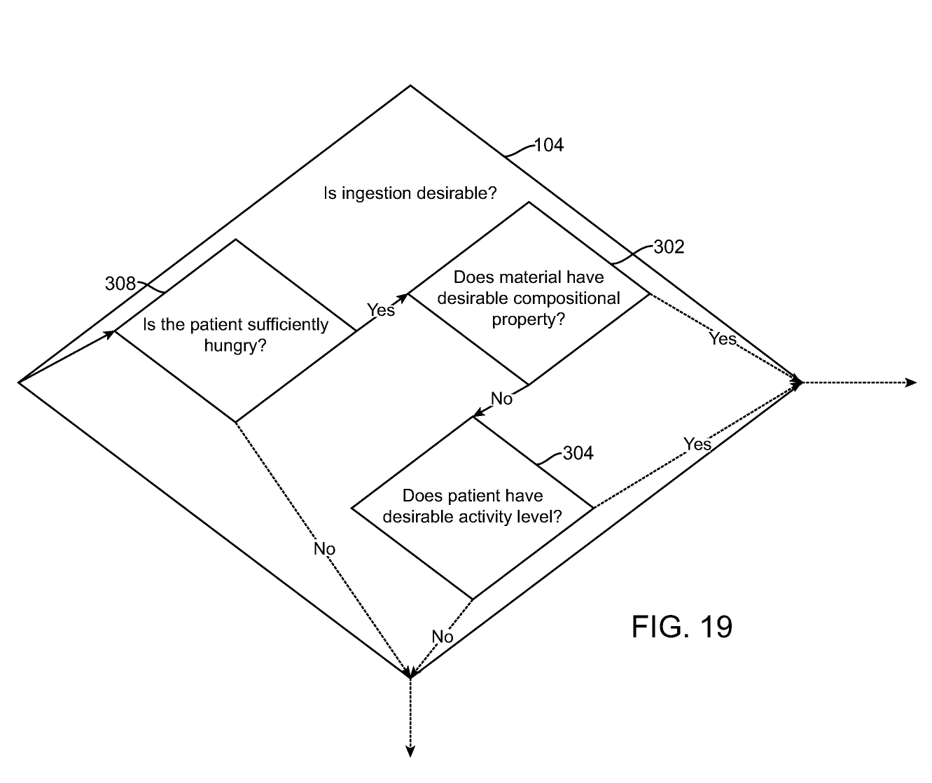

FIG. 19 illustrates an example wherein the determination of whether ingestion is desirable (step 104) comprises the determination of whether the patient is sufficiently hungry (step 308), and the optional combination of the determination of whether the material has a desirable compositional property (step 302), and further the optional combination of the determination of whether the patient has a desirable activity level (step 304). After it has been positively determined that material has been ingested (step 100), the processor then executes the module to determine whether the patient is sufficiently hungry (step 308). If this is a negative determination, the ingestion is considered undesirable and the patient is provided stimulation at or above the SET (step 106), per FIG. 3. Thus, eating while not sufficiently hungry is undesired regardless of other conditions.

If this is a positive determination (i.e. the patient is sufficiently hungry), the processor then executes the module to determine if the ingested material has a desirable compositional property (step 302). If so, the ingestion is considered desirable and the patient is provided with no stimulation or stimulation below the SET (step 108) per FIG. 3. If not, the processor then executes the module to determine whether the patient has a desirable level of activity (step 304). If so, the ingestion is considered desirable and the patient is provided with no stimulation or stimulation below the SET (step 108) per FIG. 3. If not, the ingestion is considered undesirable and the patient is provided stimulation at or above the SET (step 106), per FIG. 3. Thus, if the patient has a desirable activity level, the material may be consumed regardless of the desirability of a compositional property. However, desirable activity level does not override lack of hunger. It may be appreciated that combinations of any complexity may be used to determine desirability of ingestion.

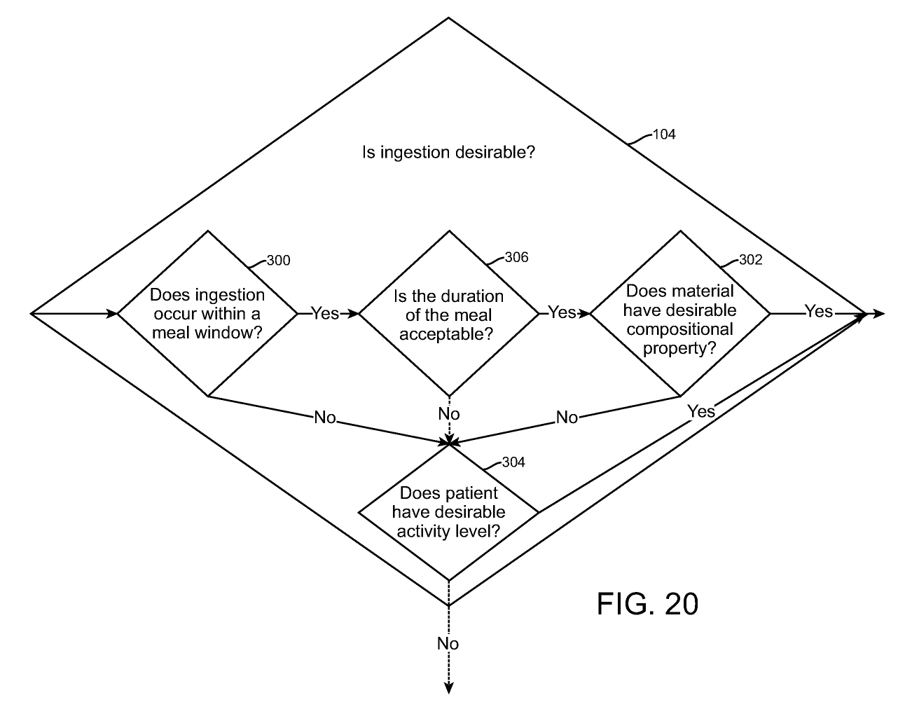

For example, FIG. 20 illustrates another example of a complex combination of determinations to determine desirability of ingestion (step 104). Here the determination of whether ingestion is desirable (step 104) comprises the determination of whether the ingestion occurs within a meal window (step 300), the optional combination of determining if the duration of the meal is acceptable (step 306), and further the optional combination of whether the material has a desirable compositional property (step 302) and whether the patient has a desirable activity level (step 304).

After it has been positively determined that material has been ingested (step 100), the processor then executes the module to determine whether the ingestion occurs within a meal window (step 300). If so, the processor then executes the module to determine if the duration of the meal is acceptable (step 306). Such combination may be useful in situations wherein the meal window is quite large. For example, the patient may be allowed a 2 hour meal window but may only be allowed 20 minutes to eat the meal. This allows the patient flexibility in planning time for a meal yet provides for binge control once the meal has commenced. If the duration of the meal is determined to be acceptable, the processor then executes the module to determine whether the material has a desirable compositional property (step 302). If this also has a positive determination, the ingestion is considered desirable and the patient is provided with no stimulation or stimulation below the SET (step 108) per FIG. 3.

If the determination was negative for any of steps 300, 306, 302, the processor then executes the module to determine if the patient has a desirable activity level (step 304). If so, the ingestion is considered desirable and the patient is provided with no stimulation or stimulation below the set (step 108) per FIG. 3. Thus, regardless of meal windows, meal duration and material composition, a patient can override these decisions, in this example, with a desirable activity level, such as exercise. However, if the activity level is determined to be undesirable, the patient is provided stimulation at or above the SET (step 106), per FIG. 3.

### Other Sequences

The above described embodiments involve determining a level of stimulation based on the determination of ingestion and the determination of desirability of ingestion. Other embodiments are provided wherein determining the level of stimulation is based on other determinations.

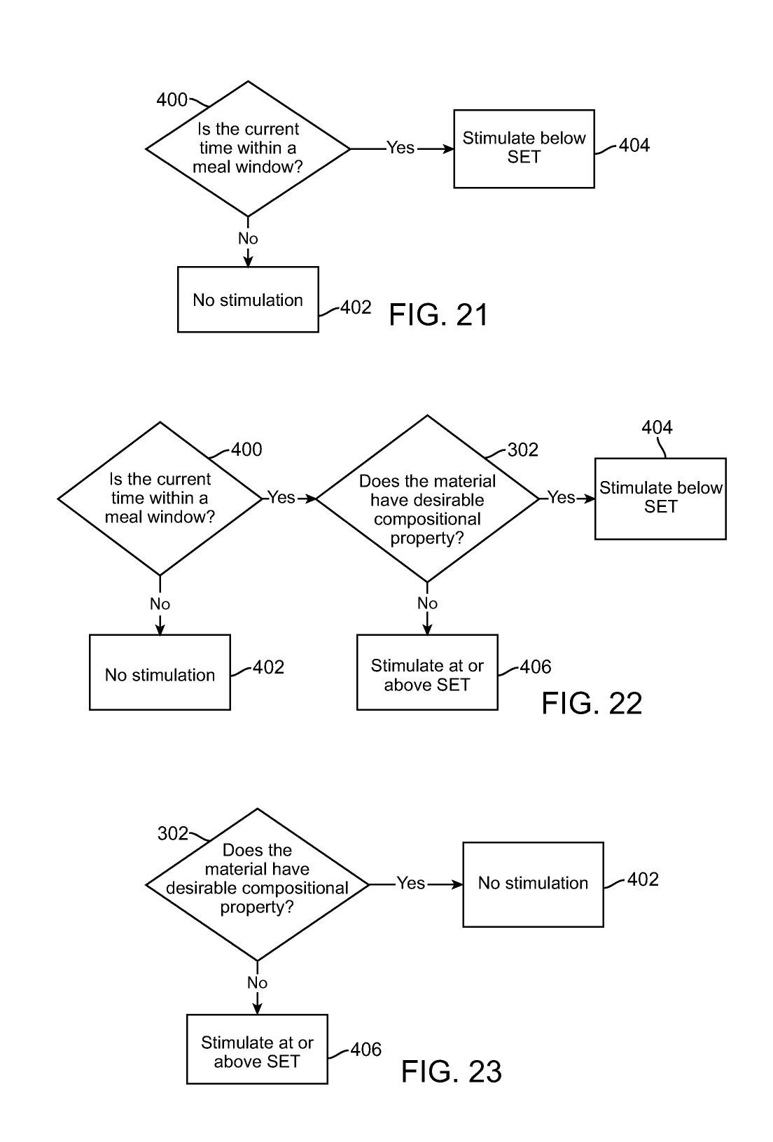

For example, FIG. 21 illustrates an embodiment wherein the determination of the level of stimulation is based on the determination of whether the current time is within a meal window (step 400). Thus, stimulation does not rely on determining if material has been ingested. Instead, the processor executes a module in the memory device which determines if the current time is within a meal window (e.g. with the use of a real time clock). At any time outside of a meal window, no stimulation is provided to the patient (step 402). Once the meal window has begun, the patient is stimulated at a level below the SET (step 404). Thus, while the patient is eating, the patient's total eating is curtailed due to the stimulation. Such an embodiment may be useful for patients in controlled eating environments wherein meals are provided at designated times. Such an embodiment may also be useful for patients who have gained a level of self-regulation and simply desire assistance during meals.

Another example, illustrated in FIG. 22, is also based on the determination of whether the current time is within a meal window (step 400). Here, again at any time outside of a meal window, no stimulation is provided to the patient (step 402). Once the meal window has begun, the processor executes a module to determine if material consumed has a desirable compositional property (step 302). If so, the patient is stimulated at a level below the SET (step 404). If not, the patient is stimulated at a level at or above the SET (step 406). Thus, the patient is deterred from eating unhealthy or undesired food during the meal window but can continue eating compositionally desirable food with stimulation control.

FIG. 23 illustrates an embodiment which does not rely on meal windows or determinations of whether material has been ingested. Here, the patient is monitored for compositional desirability of ingested material. Such monitoring may be achieved with the use of any of the sensors or devices described above to determine compositional desirability. Monitoring may be continuous or in intervals. When material having an undesirable compositional property is detected, stimulation is provided at a level at or above the SET (step 406). At all other times, no stimulation (step 402) is provided. Such an embodiment may be useful for patients wishing to improve their dietary food choices, rather than regulating quantity and timing of intake.

### Levels of Stimulation

As described above, a “stop eating threshold” or SET is established for each patient. The SET is the level of stimulation that typically causes the patient to stop ingesting. This is typically due to a displeasurable sensation, such as gastric discomfort. The actual symptoms may vary but may include nausea, pain or vomiting. In some instances, lesser symptoms may cause a cessation of eating, including but not limited to dyspepsia, fullness, bloating, etc. In some embodiments, the stimulation is varied by pulse width and amplitude until the patient becomes symptomatic. In patients that are initially responsive to a given pulse width, the patient typically becomes more symptomatic as amplitude is increased while the pulse width remains constant. In such instances, the amplitude is increased until the patient stops ingesting, therefore establishing the SET. The following data set shows two examples wherein pulse amplitude and pulse duration are paired appropriately to establish a SET:

| | Stimulation Scenario #1 | | Stimulation Scenario #2 | |
|---|---|---|---|---|
| Pulse width | 0.3 | msec | 1.0 | msec |
| Amplitude | 9 | mA | 14 | mA |

A SET may be attained with many combinations of pulse amplitude and pulse duration. In general, shorter pulse widths may require a higher pulse amplitude to establish a SET and longer duration pulse widths may require a lower pulse amplitude to establish a similar SET. In some embodiments, the amplitude is in the range of approximately 1-16 mA or approximately 1-20 mA. And in some embodiments, the pulse width is in the range of approximately 50-1000 μseconds. It may be appreciated that the SET may be alternatively or additionally established by other aspects of the stimulation signal. For example, pulse frequency, burst length, burst cycle (i.e. time on vs. time off), waveform composition (e.g. ramping up or ramping down a variable such as amplitude), etc.

Once the SET is established for each patient, the SET is stored in the memory device and utilized by the processor to provide stimulation to the patient. If by chance the patient does not respond appropriately to the SET once the gastric stimulator is in use, the stimulation level may be increased until the desired result is achieved (e.g. cessation of eating). Such increase may be gradual so as to reach the minimum stimulation level that causes the desired result. In some embodiments, the increased SET may be stored in the memory to replace the previous SET. This may overcome any adaptation or changes in patient response over time. A history of SET thresholds may be stored in the device to track the potential changes in this parameter. Historical tracking of the SET may be valuable in understanding a patient's potential adaptation to long-term gastric stimulation. Further, analysis of SET thresholds over time may reveal an association between certain patient conditions such as diabetes or other co-morbidities and the change in SET thresholds over time. This information may further fine-tune the patient selection process to identify those patients best suited for long-term gastric stimulation therapy.

As described above, the patient may be stimulated at a level below the SET to curtail consumption, such as during a meal. In some embodiments, this stimulation level has a signal with the same pulse width as the SET and an amplitude reduced by a percentage, such as approximately 10-50%, particularly approximately 25%. This level of stimulation may reduce the food consumed by weight at a meal by a percentage, such as 5-50%. Stimulation at this level typically causes the patient to feel full sooner, curtail eating time and therefore typically eat less. In some instances, such stimulation may also suppress eating at other times of day when no stimulation is provided. Thus, the effects of such stimulation may be more global and far reaching for some patients leading to successful weight loss and more healthy eating habits.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. A system for use in providing gastric stimulation to a patient, the system comprising:
an ingestion sensor;
a stimulator; and
a processor coupled to the sensor and the stimulator, the processor configured to determine
an ingestion of material by the patient,
a desirability of the ingestion by the patient, and
a level of stimulation based on the determination of ingestion and the determination of desirability of ingestion,
the processor inducing the stimulator to transmit the level of stimulation.

2. A system as in claim 1, wherein the ingestion sensor comprises a temperature sensor.

3. A system as in claim 1, wherein the processor comprises a module for determining the level of stimulation, wherein the module for determining the level of stimulation selects a level of no stimulation in response to a determination that material has been ingested by the patient and a determination that ingestion by the patient is desirable.

4. A system as in claim 3, wherein the module for determining the level of stimulation selects a level of stimulation below a personal threshold for the patient in response to a determination that material has been ingested by the patient and a determination that ingestion by the patient is desirable.

5. A system as in claim 3, wherein the module for determining the level of stimulation selects a level of stimulation at or above a personal threshold for the patient in response to a determination that material has been ingested by the patient and a determination that ingestion by the patient is undesirable.

6. A system as in claim 5, wherein the module for determining the level of stimulation includes code for increasing the level of stimulation until a desired response is given by the patient.

7. A system as in claim 1, wherein the processor comprises a module for determining the desirability of ingestion by the patient that includes a module for determining if ingestion occurs during a meal window.

8. A system as in claim 7, further comprising a real time clock.

9. A system as in claim 8, wherein the real-time clock is adjustable by a global positioning system.

10. A system as in claim 1, wherein the processor comprises a module for determining the desirability of ingestion by the patient that includes a module for determining whether the material has a desirable compositional property.

11. A system as in claim 10, wherein the sensor comprises a compositional sensor configured to sense the compositional property of the ingested material.

12. A system as in claim 1, wherein the processor comprises a module for determining the desirability of ingestion by the patient that includes a module for determining if the patient has a desirable activity level.

13. A system as in claim 12, further comprising a motion sensor configured to sense motion of the patient or sense position of the patient.

14. A system as in claim 1, wherein the processor comprises a module for determining the desirability of ingestion by the patient that includes a module for determining if the duration of the meal is acceptable.

15. A system as in claim 1, wherein the processor comprises a module for determining the desirability of ingestion by the patient that includes a module for determining if the patient is sufficiently hungry.

16. A system as in claim 15, further comprising a pH sensor, pressure sensor, mechanical sensor, or a biochemical sensor.

17. A system for use in providing gastric stimulation to a patient, the system comprising:
a stimulator; and
a processor coupled to the stimulator, the processor configured to determine
if current time is within a meal window, and
a level of stimulation based on the determination of whether the current time is within the meal window, the level of stimulation being below a stop eating threshold for the patient in response to a determination that the current time is within the meal window,
the processor inducing the stimulator to transmit the level of stimulation.

18. A system as in claim 17, further comprising a real time clock configured to provide current time.

19. A system as in claim 18, wherein the real-time clock is adjustable by a global positioning system.

20. A system as in claim 18, wherein the processor comprises a module for determining if current time is within a meal window, wherein this module compares the current time to a predetermined meal time schedule.

21. A system as in claim 17, wherein the processor comprises a module for determining the level of stimulation, wherein the module for determining the level of stimulation selects a level of stimulation below the stop eating threshold for the patient in response to a determination that ingested material has a desirable compositional property and that the current time is within the meal window.

22. A system for use in providing gastric stimulation to a patient, the system comprising:
a compositional sensor configured to sense a compositional property of ingested material;
a stimulator; and
a processor coupled to the stimulator, the processor configured to determine
desirability of the compositional property of the ingested material, and
a level of stimulation based on the determination of the desirability of the compositional property,
the processor inducing the stimulator to transmit the level of stimulation.

23. A system as in claim 22, wherein the processor comprises a module for determining the level of stimulation, wherein the module selects a level of stimulation at or above a stop eating threshold for the patient in response to a determination that the ingested material has an undesirable compositional property.

24. A system for use in providing gastric stimulation to a patient, the system comprising:
a processor; and
a memory coupled to the processor, the memory configured to store a plurality of code modules for execution by the processor, the plurality of code modules comprising:
a module for determining if material has been ingested by the patient,
a module for determining desirability of ingestion by the patient, and
a module for determining a level of stimulation based on the determination of ingestion and the determination of desirability of ingestion.

25. A method for gastric stimulation of a patient, the method comprising:
determining if material has been ingested by the patient;
determining desirability of the ingestion by the patient;
determining a level of stimulation based on the determined ingestion and the determined desirability of ingestion; and
applying the determined level of stimulation to the patient from a stimulator implanted in the patient.
